(19) United States
(12) Patent Application Publication
(10) Pub. No.: US 2008/0103362 A1
(43) Pub. Date: May 1, 2008

(54) PROGRAMMABLE BRAKE CONTROL SYSTEM FOR USE IN A MEDICAL DEVICE

(75) Inventor: Lucien Alfred Couvillon JR., Concord, MA (US)

Correspondence Address: KLARQUIST SPARKMAN, L.L.P.;MICHAEL P. GIRARD, 121 S.W. SALMON STREET, SUITE 1600, PORTLAND, OR, 97204, US

(73) Assignee: SciMed Life Systems, Inc.

(21) Appl. No.: 11/966,904

(22) Filed: Dec. 28, 2007

Related U.S. Application Data
(63) Continuation of application No. 10/955,932, filed on Sep. 30, 2004.

(51) Int. Cl. A61B 1/00

(52) U.S. Cl. 600/148

(57) ABSTRACT

A system and method for providing programmable brake control in a fly-by-wire medical instrument system are provided. In one embodiment, the invention provides a brake control system that includes a brake control algorithm that provides temporal and spatial control of the motion of a medical instrument.

### FIELD OF THE INVENTION

The present invention generally relates to medical devices and in particular to a braking system for a medical device.

### BACKGROUND OF THE INVENTION

It has become well established that there are major public health benefits from regular endoscopic examinations of a patient's internal structures such as the alimentary canals and airways, e.g., the esophagus, stomach, lungs, colon, uterus, urethra, kidney, and other organ systems. Endoscopes are also commonly used to perform surgical, therapeutic, diagnostic, or other medical procedures under direct visualization. Conventional endoscopes generally include an illuminating mechanism such as a fiber optic light guide connected to a proximal source of light, or light emitting diodes (LEDs) positioned at the distal tip of the endoscope and an imaging means such as an imaging light guide to carry an image to a remote camera or eye piece, or a miniature video camera within the endoscope itself. In addition, most endoscopes include one or more working channels through which medical devices such as biopsy forceps, snares, fulguration probes, and other tools may be passed in order to perform a procedure at a desired location in the patient's body.

Flexible endoscopes incorporate an elongated flexible shaft and an articulating distal tip to facilitate navigation through the internal curvature of a body cavity or channel. Navigation of the endoscope through complex and tortuous paths is critical to success of the examination with minimum pain, side effects, risk, or sedation to the patient. To this end, modern endoscopes include means for deflecting the distal tip of the scope to follow the pathway of the structure under examination, with minimum deflection or friction force upon the surrounding tissue. In a conventional endoscope design, mechanical control of the deflectable tip is exerted via control cables similar to bicycle brake cables that are carried within the endoscope body in order to connect a flexible portion of the distal end to a set of control knobs at the proximal endoscope handle. The examiner mechanically steers the distal tip of the endoscope to a region of interest by manipulating the control knobs. The control knobs can be locked in place once a desired position is gained. While manually turning the control knobs, the examiner receives direct feedback regarding the force required to change the position of the tip. However, common operator complaints about traditional endoscope systems include the limitations of the motion control systems which may be clumsy and non-intuitive and do not provide the ability to make fine adjustments to the position of the endoscope.

A fly-by-wire endoscope system allows an examiner to operate the motion of the distal tip of the endoscope through an input device, such as a joystick, that sends electrical signals to a processor and an actuator, such as a servo motor. While a fly-by-wire system allows for enhanced motion control through the use of servo motor parameters, the operator may no longer receive direct feedback regarding the force required to change the position of the endoscope. Adequate speed control is also important for variable resistance force for slide-by procedures in which the endoscope is drawn across a region in order to palpate or assist in navigation around bends. Therefore, in order to further enhance the safety and utility of a fly-by-wire endoscope, there is a need for a system that provides adequate speed control and is responsive to the force required to change the position of the endoscope. Such a system would also allow for a superior interface with the operator, improved access by reduced frictional forces upon the lumenal tissue, increased patient comfort, and greater clinical productivity and patient throughput than those that are currently available.

### SUMMARY OF THE INVENTION

To address the problems associated with conventional medical instrument systems, the present invention provides a programmable brake control system for a fly-by-wire medical instrument control system. The instrument control system includes a user input device and a motion processor that receives position commands from the user input device. The motion processor directs position commands to one or more motors that apply tension to control cables in a medical instrument. A programmable brake control filters the position commands with reference to the history of the instrument's position and applies filtered position commands to the one or more motors. The position commands may also be filtered as a function of one or more operating parameters of the instrument. In some embodiments, the operating parameters include the position of the instrument and its time history. In some embodiments, the instrument includes an imaging sensor, and the operating parameters include the position of the instrument as compared to a tissue wall in a patient. In numerous embodiments, the operating parameters include a procedural mode of the instrument. In a preferred embodiment, the instrument is an endoscope with a deflectable distal tip.

In another aspect, the invention is a method of providing programmable brake control in a fly-by-wire medical imaging system. The method includes obtaining input information associated with a procedural mode of the medical imaging system and determining a preprogrammed braking algorithm associated with the procedural mode. The braking algorithm is sent as a brake command data set to a motion processor. The brake command contains servo parameters that spatially and temporally control the motion of the imaging device. Motion commands to be provided to actuators within the medical imaging system for moving the distal tip of a device are filtered with the preprogrammed braking algorithm to generate modified position commands which modify the execution of the motion commands in an acutator.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram illustrative of a fly-by-wire endoscopic control system having programmable brake control in accordance with one embodiment of the present invention;

FIG. 2 is a functional block diagram of an endoscopic brake control system that shows the operational interrelationship of the major hardware and software elements of the system, in accordance with one embodiment of the present invention;

FIG. 3 illustrates one embodiment of a user input device for use with an endoscopic brake control system of the present invention;

FIG. 4 is a block diagram of a programmable brake control system showing illustrative operating parameters that are input into a brake control algorithm, in accordance with one embodiment of the present invention;

FIG. 5A graphically illustrates a brake control algorithm for a sticking friction brake force;

FIG. 5B graphically illustrates a brake control algorithm for a viscous friction brake force;

FIG. 5C graphically illustrates a brake control algorithm for an aerodynamic drag force;

FIG. 6A graphically illustrates a scalar brake force;

FIG. 6B graphically illustrates a vector brake force;

FIG. 6C graphically illustrates a brake force corresponding to a position in a three-dimensional image;

FIG. 7 graphically illustrates the response of an endoscope corresponding to input from a user input device that is modified with a brake control algorithm, in accordance with one embodiment of the present invention; and

FIG. 8 is a flow diagram of a process for providing programmable brake control based on a procedural mode of an endoscope system in accordance with an embodiment of the present invention.

### DETAILED DESCRIPTION OF THE PREFERRED EMBODIMENT

In traditional motion control systems, the control of position and velocity of an object is accomplished mechanically through physical cams, gears, shuttles, hydraulic and pneumatic cylinders and the like. With the emergence of computers and microprocessor technology, an electronic based “fly-by-wire” control system may be used in which one may choose a variety of different parameters by changing the software within the system. A programmable brake control in accordance with one embodiment of the present invention for use in a fly-by-wire medical instrument system is achieved through the application of programmable hardware and software, in conjunction with input control devices, actuators, sensors and other feedback devices, for the control of the linear and rotary motion of the distal tip of the endoscope. The brake control system of the present invention allows for incremental and responsive temporal and spatial control of the motion of the distal tip of a fly-by-wire endoscope by providing programmable brake control algorithms through which position commands are processed to modify the position, speed and/or rotation of the distal tip of the endoscope as a function of one or more operating parameters of the endoscope. The system of the invention therefore allows a high level of interactive brake control that is responsive to various operating parameters of an endoscope. The operating parameters of the endoscope include various procedural modes of the system, parameters determined from feedback sensors (e.g., analysis of images received from a patient's body, torque, position and the time-history of position of the servo motors that drive the device) and patient specific parameters (e.g., sex, age, medical history, and the like). In some embodiments, the brake control system automatically responds to input operating parameter signals and sends spatial and/or temporal signals to the servo processor/controller to accelerate or decelerate the distal tip based on a set of programmed parameters. In other embodiments, the input operating parameter signals are supplied by a user via a user input device.

It will be understood by one skilled in the art that while the invention is described in reference to an endoscope with a control device that controls the deflection of the distal end of the shaft, the programmable brake control system and methods of the invention may be used in any medical instrument system that includes a steerable device and a control device.

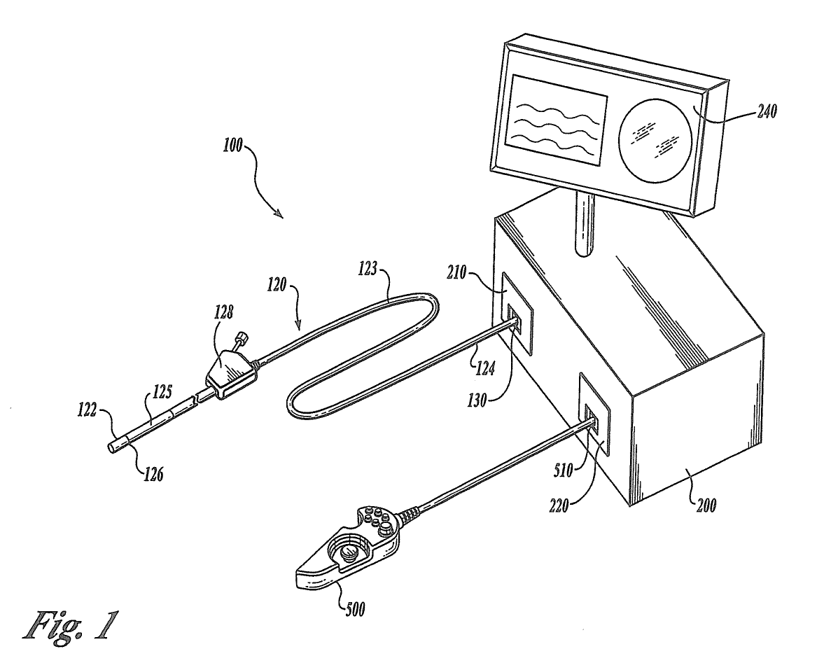

FIG. 1 illustrates the major components of an exemplary fly-by-wire endoscopic imaging system 100 having programmable brake control according to the present invention. The components of the system 100 include an endoscope 120, comprising a shaft 123 having a distal end 125 and a proximal end 124. The distal end 125 includes a tip 122 having an imaging element (not shown) and the proximal end 124 has a connector 130 that is attachable to a control unit 200. Proximal to the distal tip 122 is an articulation joint 126 that provides sufficient flexibility to the distal section of the shaft such that the distal tip 122 can be directed over the required deflection range (180° or more) by the steering mechanism and can be directed to make that bend in any direction desired about the circumference of the distal tip 122. The endoscope 120 has a set of control cables (not shown) that control the motion of the distal tip 122. The ends of the control cables are attached at or adjacent the distal tip 122, and run the length of the endoscope 120 while the proximal ends are connected to actuators in the control unit 200.

In the embodiment shown in FIG. 1, the endoscope 120 also includes a breakout box 128 that is positioned approximately midway along the length of the shaft 123. The breakout box 128 provides an entrance to a working channel and may include additional access points to lumens in the scope for collection of samples and surgical manipulation. The endoscope system 100 also includes a user input device 500 that is functionally connected to the control unit 200. The control unit 200 executes application software residing therein comprising position and brake force control algorithms to provide linear or nonlinear temporal and spatial control of the motion of the distal tip 122 as described in more detail below. The control unit 200 also includes a medical device interface 210, a user input device interface 220 and a display 240. The user input device 500 is attachable via a wired or wireless connection 510 to the control unit 200.

In operation, a physician (or other medical person) first advances the distal tip 122 of the endoscope 120 into a patient's body cavity. The physician then may use the user input device 500 to input control signals to the control unit 200 to direct the motion of the distal tip 122 of the endoscope 120. As will be explained in further detail below, the user input device 500 is capable of sending a variety of motion control signals to the control unit 200, including steering, orientation and brake control signals that control the motion of the distal tip 122.

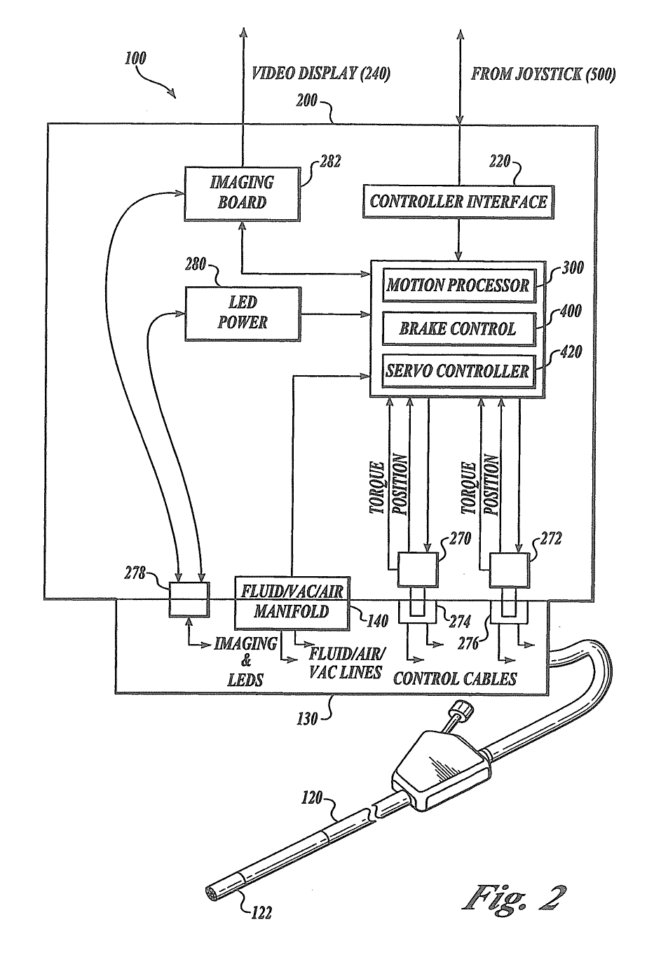

FIG. 2 is a functional block diagram of one embodiment of an endoscopic imaging system 100 with a brake control system of the present invention. The system 100 includes the control unit 200 that operates to control the orientation, steering and braking functions of the distal tip 122 of the endoscope 120. The control unit 200 includes a user input device interface 220 that connects the control unit 200 to the user input device 500. Control commands from the user input device 500 are supplied to a motion processor 300 such as a digital signal processor. In the embodiment shown, the motion processor 300 sends position commands to a servo controller 420 that controls the operation of a pair of servo motors 270, 272 which, in turn, rotate drive shafts 274, 276 coupled to control cables within the endoscope 120 in order to control the motion of the distal tip 122. Prior to execution of the position commands in the servo controller 420, the position commands are modified by a brake control 400 that filters the position commands as a function of one or more endoscope operating parameters. Although the embodiment shown in FIG. 2 shows two servo motors and four control cables, it will be appreciated that additional servo motors and fewer or more control cables could be used to move the distal tip. Further, although the disclosed embodiment uses rotary servo motors to drive the control cables, other actuators such as linear actuators could be used.

With continued reference to FIG. 2, the endoscope 120 is attached to the control unit 200 via the connector 130. The connector 130 includes an imaging interface 278, a fluid/vacuum/air manifold 140 that is controlled on the control unit 200 to selectively deliver insufflation gas, irrigation liquids and vacuum to the lumens of the endoscope (as disclosed in U.S. patent application Ser. No. 10/811,781, filed Mar. 29, 2004, and incorporated by reference) and a continuation-in-part application entitled VIDEO ENDOSCOPE, filed Sep. 30, 2004, and identified by Attorney Docket No. BSEN123550. An imaging board 282 is included in the control unit 200, along with an illumination power source 280 to power the LEDs at the distal end of the endoscope. An imaging interface 278 in the connector 130 receives signals from the image sensor in the endoscope and supplies them to the imaging board 282. The imaging board 282 produces images that are sent to a video display 240. The imaging board 282 is also capable of analyzing images of tissues to determine information such as, for example, the thickness of the tissue wall as a function of the illumination intensity, or the position of the distal tip in comparison to a tissue wall as described in more detail below. The information from the imaging board is provided as one type of operating parameter that can be used in the brake control algorithm 400 as discussed in more detail below.

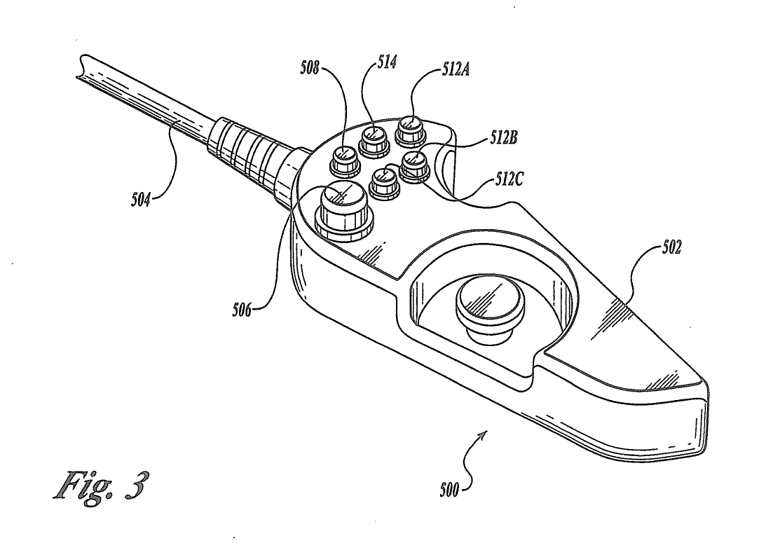

FIG. 3 illustrates one embodiment of the user input device 500 configured as a handheld controller. The user input device 500 includes a body 502 that may be coupled to the control unit 200 via an electrical cord 504, a wireless radio frequency channel, an infrared or other optical link. In the fly-by-wire endoscopic imaging system 100, the user input device 500 produces electrical control signals that are delivered to the control unit 200. Positioned in an ergonomic arrangement on the user input device 500 are a number of electrical switches. An articulation joystick 506 or other multi-positional device can be moved in a number of directions to allow the physician to steer the distal tip 122 of the endoscope 120 in a desired direction. In some embodiments, the physician guides the endoscope remotely by moving the joystick 506 while watching an endoscopic image on the video monitor 240 or by viewing the position of the distal tip 122 with another medical imaging technique such as fluoroscopy.

With continued reference to FIG. 3, a camera button 508 is provided to capture an image of an internal body cavity or organ in which the endoscope 120 is placed. The captured images may be still images or video images. The images may be adjusted for contrast or otherwise enhanced prior to display or stored in a recordable media. The user input device 500 also includes at least one brake button 514 that allows a physician to apply a variable brake function to slow or stop the motion of the distal tip 122, or to preserve the position of the distal tip 122. In some embodiments, one or more additional brake buttons 512A, 512B, 512C may also be provided to allow a physician to apply various brake control functions as further discussed below. Additional buttons may be added to the user input device 500 to activate additional functions such as irrigation, insufflation, vacuum control and the like.

In one embodiment of the invention, the joystick 506 on the user input device 500 initiates a position-to-rate control implemented by the motion processor 300 and brake control 400 that varies the speed at which the distal tip 122 is moved as a function of the joystick 506 position. In other embodiments, other position control algorithms including position-to-position or position-to-force (i.e., acceleration) are implemented using the joystick 506. In some embodiments, each position control command initiated by the user input device 500 corresponds to a procedural mode with a corresponding brake control as further discussed below.

In some embodiments, the controller 500 also includes a force feedback mechanism (not shown) that applies a variable force to a spring or other such equivalent structure that biases the joystick 506 that the user uses to position the endoscope in response to forces on the endoscope. Therefore, the user is given a tactile indication of the force required to steer the endoscope in the patient's body. U.S. application Ser. No. 10/811,781, filed Mar. 29, 2004, and a continuation-in-part application entitled VIDEO ENDOSCOPE, filed Sep. 30, 2004, and identified by Attorney Docket No. BSEN123550 discloses various mechanisms for varying the feedback force on a joystick in proportion to the torque required to steer the endoscope and/or the amount of articulation at the distal tip.

In operation, control commands from the user input device 500 are sent to the motion processor 300 which executes a motion control program to convert the signals received from the user input device 500 into position commands that control the amount of tension applied to the control cables within the endoscope. To execute delivery of the position commands by the servo controller 420, the motion processor 300 follows a brake control algorithm 400 that filters the position commands as a function of various endoscope operating parameters to produce modified position commands. The modified position commands are then sent to the servo controller 420 that controls servo motors 270, 272 which in turn selectively tension or release the control cables in the endoscope 120 to control the orientation of the distal tip.

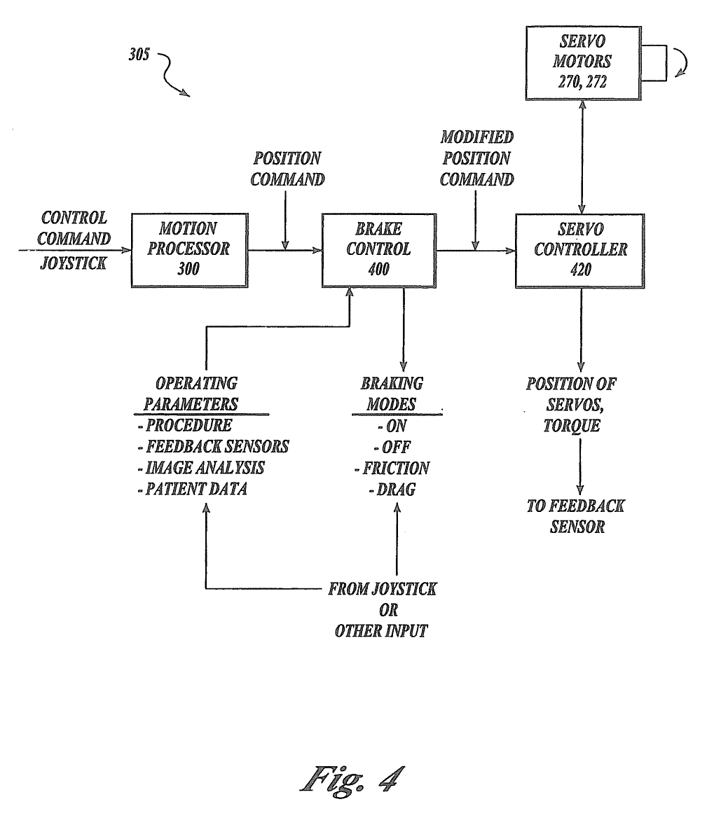

As shown in FIG. 4, the brake control algorithm 400 receives input regarding various endoscope operating parameters. Operating parameter input may be received from the user input device 500, feedback sensor 326, imaging board 282 or other source. For example, the brake control algorithm 400 may receive input regarding the procedure currently in use in the endoscope system, such as a steering mode, an examination mode or a surgical mode. Feedback sensors, such as feedback sensors 326 and other feedback sensors associated with the servo motor or positioned within the endoscope provide input parameter information regarding, for example, the position of the servo motors, the velocity of the distal tip of the endoscope, and/or the torque required to move the control cables. Alternatively, feedback signals may be stored in a memory to produce a history, moving average, peak, minimum or other statistical calculation of the velocity, acceleration, torque or position of the distal tip of the endoscope.

Image analysis information provided from the imaging board is another operating parameter that may be used in the brake control algorithm 400. In some embodiments, the endoscope has an imaging sensor with an illuminating mechanism such as a fiber optic light guide or an LED. In such embodiments, input sensory feedback may be provided to the brake control algorithm 400 from the imaging sensor and the imaging board 282. For example, the relative position of the endoscope tip in comparison to a tissue wall can be determined by a physician using a visual image obtained from the image sensor. Alternatively, the brightness and/or area of an illuminated region of a tissue wall can be used to determine the proximity of the imaging sensor to the tissue wall. Alternatively, a sensor such as an ultrasound transmitting receiver may be positioned in the distal tip to provide signals indicative of the thickness of the tissue, or the relative location of the endoscope tip in comparison to a reference point in the patient's body.

An additional operating parameter that may be used in the brake control algorithm 400 is a measurement indicating the elasticity of a patient's tissue in the vicinity of the distal tip of the endoscope. An estimate of tissue elasticity in the vicinity of the distal tip can be made by means of measuring the result of a stimulated response. For example, a test can be made by sending a motion command to the motion processor 300 to actuate a small perturbing test force or insufflation pressure to exert force to a tissue wall. The dynamic deflection of the distal tip is then measured from which the level of tissue elasticity is inferred. A value representing the tissue elasticity in the vicinity of the distal tip is then used as an operating parameter in the brake control algorithm 400.

An estimation of the three-dimensional shape of the endoscope with regard to the coiling of the shaft can be made and used as an additional operating parameter in the brake control algorithm 400 to adjust for capstan friction losses. The shape of the flexible elongated endoscope shaft in a patient's body at a particular point in time may be modeled as a series of coiled loops. The amount of coiling of the loops affects the gain of the control cables due to capstan friction losses. Various sensors can be used to measure the coiling of the loops. For example, a string of deflection gauges placed upon the scope along its length can be used to measure the extent of coiling. In another example, an array of electromagnetic sensors may be incorporated along the length of the scope that communicate with localizing coils, such as in a goniometer. In yet another example, an assessment of the electrical impedance at the driving point of a conductor built into the endoscope can be used to infer the level of coiling of the loops. A value representing the extent of three-dimensional coiling of the endoscope shaft is then used as an operating parameter in the brake control algorithm 400 to adjust for capstan losses.

In some embodiments, the programmable brake control algorithm 400 utilizes input operating parameter data regarding patient specific information. In accordance with this embodiment of the invention, operating parameters associated with a particular patient are entered into the patient parameter database 318 through a user interactive device such as a keyboard connected to the control unit 200 (not shown). For example, the user may be prompted to enter the type of procedural mode(s), download images previously associated with the patient and enter other relevant characteristics of the patient such as age, weight, and the like. Operating parameters may also include the make and model of the endoscope device in use.

In some embodiments, the programmable brake control algorithm 400 utilizes one or more input operating parameters to generate an automatic brake force responsive to feedback signals regarding the velocity and/or position of the endoscope during clinical use. For example, feedback signals are generated based upon the position of the servo motors from which the length that control cables are extended/shortened is determined as well as the torque required to move the control cables. The feedback signal data is processed by the motion processor 300 and an approximation is made of the amount of articulation at the distal tip 122 of the endoscope 120. The motion processor 300 uses the brake control algorithm 400 to send a particular brake command to the motion processor 300 in response to a set of feedback parameters.

In some embodiments, the operating parameters used to determine brake force include both feedback signals from the endoscopic imaging system and user input signals from a user input device 500 controlled by the physician. The feedback signals can be displayed to the physician on the video display 240 along with the images received from the image sensor, patient data and other relevant operating parameters of the endoscope imaging system. The user input device 500 can be used to send an input signal to the brake control 400 along with the other feedback parameters to generate an appropriate brake control algorithm 400 which filters command signals from the motion processor 300 before they are executed by the servo controller 420.

In an additional embodiment, the endoscopic imaging system comprises an artificial intelligence self-learning system that remembers a user's past selections and preferences regarding the use of the brake control algorithm 400 and other operating parameters of the endoscope imaging system. In such an embodiment, the endoscopic imaging system is programmed to crosscheck a user's past selections with an operating parameter to recommend optimum settings for the brake control algorithm 400. The artificial intelligence self-learning system may be provided locally in the endoscopic imaging system, or remotely to the system via a remote server connected via a communications network.

In a further embodiment, the programmable brake control system automatically responds to endoscopic images from the endoscope imaging sensor that provides graphic indications of the approximate location of the endoscope 120 as received from the image sensor. For example, in a colonoscopy, where the endoscope 120 is advanced to the cecum, the imaging board 282 analyzes the image of the colon and compares the image to a set of pre-stored parameters and input is sent to the braking control algorithm 400 which filters the position commands as a function of the comparison to generate modified position commands which are in turn sent to the servo controller 420. The modified position command can be generated such that the distal tip 122 is oriented in the direction of the dark open lumen or so that the tip dwells on objects of interest, such as polyps, during an examination. The input signals from the imaging sensor can also be combined with feedback sensor information such as the position of the servo motors, the velocity of the distal tip of the endoscope and the torque required to move the control cables as described previously.

The brake control algorithm 400 of the present invention is programmable to provide brake commands that direct linear or nonlinear temporal and spatial control of the motion of the distal tip 122 of the endoscope 120. The brake control algorithm 400 can send a command to the motion processor 300 that implements any type of desired brake force required to properly orient the distal tip 122 of the endoscope 120. The brake command may include information specifying the time of initiation and termination of the brake force, the type of brake force to apply (e.g. a friction algorithm, a viscous drag algorithm or an aerodynamic drag algorithm), and the magnitude and direction of the braking force (expressed either in polar coordinates, or with respect to an endoscopic image). In operation, the programmable braking algorithm coupled with manual user input control allows the physician to move the distal tip with a light touch. The brake command can include parameters adjusting the servo motor gain as well as the transient response. The brake algorithm may also adjust the order of the servo parameters as well, from a simple first-order response characterized by a single time constant, to more complex servo parameters with selectable damping, overshoot, ringing, phase delay and the like.

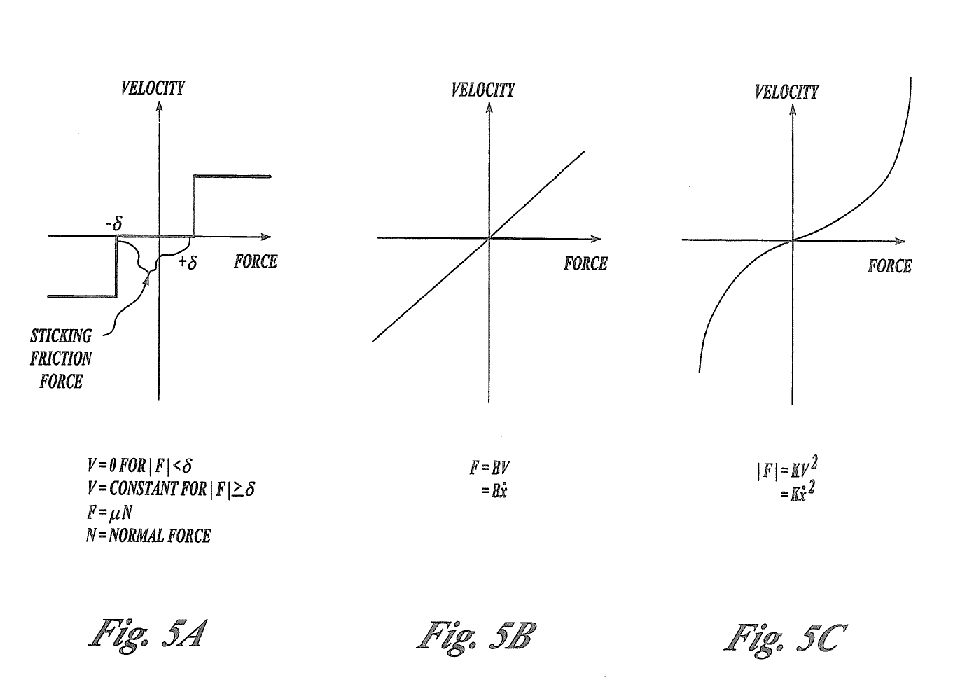

Various types of brake force can be modeled through the use of preset algorithms in the brake algorithm 400 to provide different braking force modes. For example, as shown in FIG. 5A, the braking mode may mimic a sticking friction brake where the brake force algorithm is (F=μN) where F is the brake force applied and where N is the normal force. This sticking friction brake mode would provide a constant braking force for all displacements after an initial sticking force is overcome. In another example, as shown in FIG. 5B, the brake command may represent a viscous friction brake force (F=Bv or F=B\dot{x}). This viscous friction brake algorithm would provide brake force that is proportional to the velocity at the distal tip. In yet another example, as shown in FIG. 5C, the brake command may represent an aerodynamic drag force (F=Kv²), which would make brake force proportional to the square of velocity. The brake force command may be one of these above-mentioned algorithms, or other types of brake force algorithms known to those of ordinary skill in the art of control systems. The brake force command may also be a programmed blend of brake force algorithms to achieve the best operator comfort and performance. In some embodiments, the brake force command includes additional temporal and spatial variables as further described below.

The brake force command can also include a spatial control component. Spatial control can be achieved by applying different amounts of force to individual servo motors 270, 272. The servo controller 420 can interface with and control more than one servo motor 270, 272 through the use of a single brake control algorithm 400. The brake control algorithm 400 includes a number of characterizable parameters, each of which can be independently characterized for each servo motor 270, 272 the brake control algorithm is to control. For example, one pair of control cables can drive the distal tip in the up and down directions, while the other pair can drive the distal tip in the left and right directions. The brake control algorithm 400 can independently adjust the motion commands provided to each servo motor to allow one drag force to be applied in the up and down direction and a different drag force (or no brake) to be applied in the left/right direction, thereby allowing for greater control and manipulation of the orientation of the distal tip.

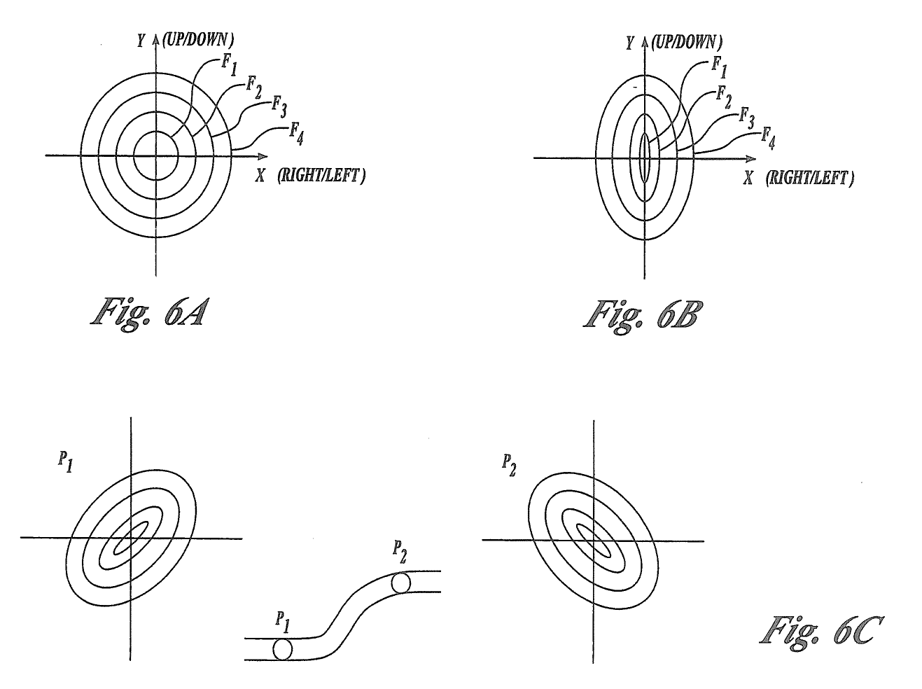

As shown in FIG. 6A, the brake parameters can be spatially applied in a scalar fashion, so that the force is the same in the up/down (y-axis) and the right/left (x-axis) direction. The contours of the magnitude and direction of force shown as polar coordinates F_{1x,y}, F_{2x,y}, F_{3x,y}, and F_{4x,y} are applied equally to each servo motor 270, 272 so that the distal tip would decelerate equally with respect to the x and y coordinates of the distal tip.

As shown in FIG. 6B, the brake force can also be spatially applied in a vector fashion, so that the distal tip responds differently in the up/down (y-axis) and in the left/right (x-axis) direction. In this situation, the contours of the magnitude and direction of force shown as polar coordinates F_{1x,y}, F_{2x,y}, F_{3x,y} and F_{4x,y} would be applied to each servo motor 270, 272 in different amounts, wherein F₁, F₂, F₃ and F₄ represent contours of constant force. For example, regarding F_{1x,y}, the F_{1y} position command to the servo motor 270 that moves the distal tip in the up/down direction would be a greater value than the F₁ₓ coordinate corresponding to the position command to the servo motor 272 that moves the distal tip in the left/right direction. Therefore, a different type and/or amount of force may be applied to each servo motor 270, 272 such that the distal tip decelerates differently with respect to an up/down and left/right motion.

The spatial control may alternatively be specified in reference to an image, such as for example, the endoscopic image from the image sensor, the shape of the endoscope as inferred from the image, or from the shape determined from position sensors. As shown in FIG. 6C, at a first position (P₁), the braking force is spatially applied based on a first control locus, as determined by the three-dimensional slope from the first image, and as the scope is advanced to a second position (P₂), the braking force is spatially applied based on a second control locus, as determined by the three-dimensional slope from the second image.

In some embodiments, the brake control algorithm 400 is an input/output system in which input operating parameter signals detected by the brake control algorithm 400 activate the brake control algorithm to send an output signal comprising a predetermined brake force command to the motion control processor 300. Input signals may be detected from a user input device or from a feedback sensory device as described previously. In accordance with this embodiment of the system, various procedural modes may be programmed into the brake control algorithm 400, wherein the particular procedure is determined from a set of operating parameters from a source such as a user input device 500. For example, the brake control algorithm 400 may interpret input signals from the user input device 500 that correspond to a change in the position of the joystick as a “steering mode” and in turn provide a predetermined output brake command that specifies a particular sticking friction brake force, thereby providing a fast brake response. In another example, input signals from the user input device 500 that correspond to a change in position of a brake button of the user device is interpreted as “observation mode” and the brake control program provides a predetermined output brake command that specifies a more viscous friction brake force, thereby providing slow deceleration to allow a physician to observe a particular region of the body. In some embodiments, the user input device 500 may contain a designated button for each of several braking modes as described previously.

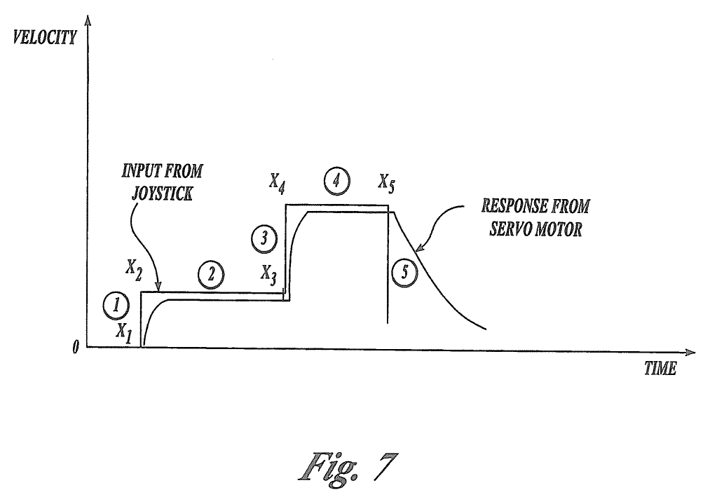

FIG. 7 graphically illustrates the input from the user input device 500 and the response of the servo motors 270, 272 actuated by the motion processor 300, braking control algorithm 400 and servo controller 420 based on control commands and brake commands from the user input device 500 and brake control program 314 as a function of velocity (shown on the y axis) versus time (shown on the x axis). As shown in FIG. 7, the initial speed of the endoscope is y=0. During segment 1, the joystick sends a control command to move the endoscope tip in a “steering mode.” The servo motor responds with a “fast response” and the brake control algorithm 400 modifies the velocity of the distal tip with a sticking friction brake force, resulting in a constant braking force after the initial sticking force is overcome. During segment 2, the joystick holds position of the distal tip and the velocity of the endoscope remains unchanged. During segment 3, the joystick sends a control command to move the endoscope tip in another “steering mode” and the servo motor again responds with a “fast response.” In segment 4, the joystick holds position of the distal tip and the velocity of the endoscope remains unchanged. Finally, in segment 5, a brake button is engaged on the user input device 500, signaling a “slow decay” mode corresponding to a viscous friction brake force so that the endoscope tip tends to dwell upon a feature of interest as previously described.

Other procedural modes may be preprogrammed into the brake control algorithm 400 such as procedural modes corresponding to insertion and removal of an endoscope from a patient, examination mode, surgical procedure modes, and the like. For example, during endoscope insertion and removal, a brake control algorithm can be applied with appropriate force to lock the distal tip in a safe position. In another example, in an examination mode, position control signals can be produced which cause the distal tip to move in a controlled spiral search pattern so that all areas of a body cavity are scanned for the presence of disease. The brake control algorithm 400 can filter these position control signals so that the movement at the distal tip stays within preset boundaries, or adapt to regional conditions such as, for example, tissue elasticity. In another example, in a surgical mode, such as taking a tissue sample from a lumen wall, the brake control algorithm 400 can be applied to maintain the position of the distal tip in a predefined orientation.

In another embodiment, the brake control algorithm 400 implements variable brake control that combines user input control with preselected braking thresholds based on various parameters such as torque, location, maximum speed, and the like. The use of preselected braking thresholds provides a safety mechanism that prevents the distal tip from exceeding certain thresholds. The variable braking threshold can be set to a low value near delicate portions of the patient's anatomy, and the brake force can be set to prohibit a rapid response, so that user control of the joystick results in fine movements of the distal tip. For example, variable brake control is accomplished by having the physician or the brake control algorithm 400 select a variable braking threshold that is between 0 and the maximum torque that can be supplied by the servo motors. When the physician moves the scope, the torque on the motors is detected to see if it is greater than or equal to the variable braking threshold. If so, the motion processor 300 and servo controller 420 applies a drag force in a pre-selected brake control algorithm that corresponds to the parameter threshold. The force is applied until the parameter, such as the torque reading, falls below the variable braking threshold.

In numerous embodiments of the programmable brake control algorithm 400, a traditional manual friction brake is also provided as a safety option to allow an operator to apply mechanical brake force to the endoscope tip.

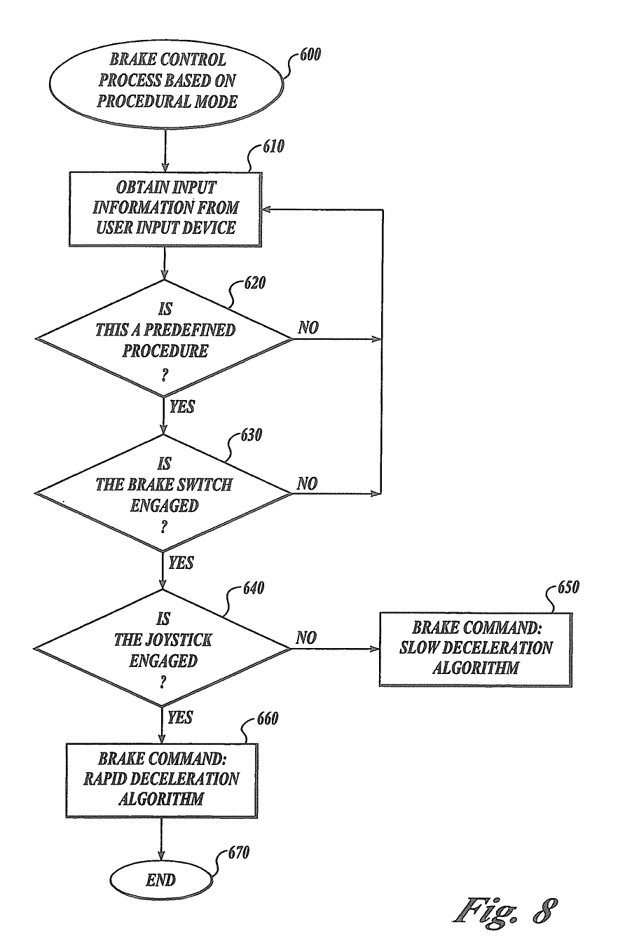

In another aspect, the present invention provides methods for providing programmable brake control in an endoscope. In some embodiments, the method provides programmable brake control based on the procedural mode of the endoscope, as determined from joystick input, selected procedure, or sensory input. FIG. 8 is a flow chart of a process for programmable brake control based on the procedural mode of an endoscope. The brake control process begins at 600 and comprises obtaining input information from a user input device associated with the endoscope at 610. A test is made at 620 to determine if the input information is recognized as a predefined procedural mode. If not, the process returns to obtain input from the user input device at 610. If the input is recognized as a predefined procedural mode, it is determined if the brake button is engaged in the handheld controller at 630. If not, the method returns to receive input from the device at 610. If the brake button is engaged at 630, another test is made at 640 to determine if the joystick is also engaged in the user input device. If not, a brake command message is sent to the servo processor that creates a slow deceleration algorithm at 650. If the joystick is engaged at 640, a brake command message is sent to the servo processor comprising a rapid deceleration algorithm at 660. Once the message is sent, the process ends at 670. In some embodiments, the machine also sends information based on input sensors with respect to spatial control parameters. The procedural mode may also be programmed as an automatic procedure as determined from input from the computer, or from interactive commands from a console, such as, for example, sample retrieval procedure, device removal and the like which would trigger a corresponding preprogrammed brake response.

While the preferred embodiment of the invention has been illustrated and described, it will be appreciated that various changes can be made therein without departing from the scope of the invention. It is therefore intended that the scope of the invention be determined from the following claims and equivalents thereof.

1-13. (canceled)

14. A control system for controlling an medical device having a shaft with a distal end, a proximal end, and one or more control cables secured at or adjacent the distal end of the shaft to selectively orient the distal end upon tension of one or more of the control cables, comprising:
user input device with which a user enters commands to orient the distal end of the medical device;
a motion processor that produces position commands in response to user inputs to the user input device;
one or more motors that selectively tension the one or more control cables to orient the distal end of the medical device in the up/down and left/right directions; and
a programmable brake control that filters the position commands produced by the motion processor with one of a number of selectable braking functions that modify the position commands produced by the motion processor to control the forces applied by the one or more motors such that the up/down and left/right response of the distal end of the shaft is independently controllable in response to the same user entered command to the input device.

15. The medical device control system of claim 14, wherein the braking function is selected in response to the torque required to move the control cables with the one or more motors.

16. The medical device control system of claim 14, wherein the braking function is selected in response to a time history of the position of the distal end of the medical device.

17. The medical device control system of claim 14, wherein the medical device further comprises an imaging sensor and wherein the position of the distal tip is measured as a function of the position of the distal end as compared to a tissue wall in a patient.

18. The medical device control system of claim 14, wherein the medical device further comprises a sensor for detecting tissue wall thickness in a patient and wherein the braking function is selected in response to the detected thickness of a tissue wall in a patient.

19. The medical device control system of claim 14, wherein the control system is operable in a number of procedural modes, and the braking function is selected according to the procedural mode of the control system.

20. The medical device control system of claim 14, wherein the braking function is selected according to information associated with a patient.

21. The medical device control system of claim 14, wherein one of the selectable braking functions filters the position commands with an algorithm corresponding to a sticking friction force.

22. The medical device control system of claim 14, wherein one of the selectable braking functions filters the position commands with an algorithm corresponding to a viscous friction force.

23. The medical device control system of claim 14, wherein the user input device includes one or more buttons with which a user selects a braking function.

24. The medical device control system of claim 14, wherein the user input device includes a joystick.
